(19) United States
(12) Patent Application Publication
(10) Pub. No.: US 2023/0270959 A1
(43) Pub. Date: Aug. 31, 2023

(54) SYSTEMS AND METHODS FOR DETECTING USAGE INFORMATION FOR A SENSOR

(71) Applicant: GE Precision Healthcare LLC, Wauwatosa, WI (US)

(72) Inventors: Russell J. Kuzelka, McFarland, WI (US); Donald L. Beduhn, Madison, WI (US)

(21) Appl. No.: 17/682,816

(22) Filed: Feb. 28, 2022

(51) Int. Cl. A61M 16/00; A61M 16/01

(52) U.S. Cl. CPC A61M 16/0051; A61M 16/01; A61M 16/0003; A61M 2016/003; A61M 2016/0027; A61M 2205/3334; A61M 2205/3368; A61M 2205/60

(57) ABSTRACT

Techniques described herein can identify usage information for sensors. In one example, an anesthesia device can include a processor to obtain usage information from a first flow sensor coupled to the anesthesia device. The processor can also determine the usage information exceeds a predetermined limit and generate an alert indicating the first flow sensor is to be replaced.

### FIELD

Embodiments of the subject matter disclosed herein generally relate to sensors and, more specifically, to detecting usage information for sensors.

### BACKGROUND

Various technologies and devices are used to measure airway gas flow and volume deliveries. These devices can include pneumotachometers, hot wire anemometers, rotating vane spirometers, and ultrasound flowmeters, among others. These devices can offer different benefits and drawbacks depending on the underlying property used to detect a gas flow.

A pneumotachometer, for example, uses a restrictor in the gas flow passage to create a pressure drop that can be sensed by a differential pressure transducer. Each output signal from the pressure transducer consistently represents a gas flow rate, and is calibrated to accurately report the measurement in gas flow rate. In some examples, an orifice is a simple and inexpensive construction for a flow restrictor. One disadvantage of a fixed orifice is its non-linear relationship between the differential pressures and the gas flow rates. The size of the fixed orifice is a compromise between a tolerable flow resistance at high flow rates, and adequate obstruction to create detectable differential pressures at low flow rates. If the selection of the orifice size favors the low flow sensitivity, the pressure transducer can experience issues with the measurement range at high gas flows. If the orifice size favors high gas flow range, the pressure transducer would not receive a detectable signal for measurement sensitivity at the low flow rates.

A compromise in measurement range also affects computation of patient tidal volumes and minute volumes, which are derived by integrating gas flows in the airway. A device that can obtain a large flow range measurement can be used to monitor gas flows for various different patients. For example, fixed orifice sensors require separate flow sensors for adult and pediatric patients. A variable orifice flow sensor can enable a single sensor for adult patients, pediatric patients, high gas flows, and low gas flows.

In some examples, tidal volumes and minute ventilation are obtained from an expiratory flow sensor in the breathing circuit for a patient. The tidal volumes and minute ventilation can be used to detect and provide alarms in response to detecting low minute ventilation and apnea. Accordingly, a variable orifice flow sensor can continuously monitor the appropriate volume delivered by the ventilator to a patient, and provide an alarm when the expired gas volume varies significantly from the setting. In some examples, such variations may be caused by leaks or issues with a valve or a variable orifice flow sensor.

Additionally, moisture is an inherent by-product of carbon dioxide absorption in the circle breathing system for a patient, especially in low flow anesthesia practice. Moisture may cause small beads of water or a foggy appearance in a flow sensor, which can affect performance. For example, pooled water in the flow sensor or water in the sensing lines can result in false readings.

In some examples, the various issues with a flow sensor, such as a variable orifice flow sensor, can arise if the flow sensor has been used to monitor the gas flows to a patient or from a patient for an extended period of time. Techniques to detect usage information indicating how long a flow sensor has been monitoring a breathing circuit for a patient are described herein.

### SUMMARY

This summary introduces concepts that are described in more detail in the detailed description. It should not be used to identify essential features of the claimed subject matter, nor to limit the scope of the claimed subject matter.

In one aspect, an anesthesia device can include a processor that can obtain usage information from a first flow sensor coupled to the anesthesia device and determine the usage information exceeds a predetermined limit. The processor can also generate an alert indicating the first flow sensor is to be replaced. The usage information can include a number of movements of a diaphragm of the first flow sensor.

In another aspect, a device can include a processor to obtain usage information from one or more flow sensors coupled to one or more anesthesia devices, determine a number of breathing cycles monitored by each of the one or more flow sensors, determine an accuracy limit value representing when the one or more flow sensors provide inaccurate flow values from an inhalation portion or an exhalation portion of a breathing circuit for a patient, and transmit the accuracy limit to the one or more anesthesia devices.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present examples will be better understood from reading the following description of non-limiting embodiments, with reference to the attached drawings, wherein below:

FIG. 1 is a block diagram of an example of a computing device that can identify usage information of a flow sensor, according to examples herein.

FIG. 2 illustrates an isometric view of a fluid flow sensor, according to examples herein.

FIG. 3 depicts an example of an inside segment of a fluid flow sensor, according to examples herein.

FIG. 4 depicts an example of an electronic vaporizer system operably connected to a ventilator system and configured to deliver vaporized anesthetic agent to a patient breathing circuit, according to examples herein.

FIG. 5 depicts an example remote device electronically coupled to an anesthesia device, according to examples herein.

FIG. 6 illustrates a process flow diagram of an example method for identifying usage information of a flow sensor, according to examples herein.

FIG. 7 is a process flow diagram of another example method for identifying usage information of a flow sensor, according to examples herein.

FIG. 8 is a process flow diagram of an example method for identifying accuracy information for a flow sensor, according to examples herein.

FIG. 9 is an example of a non-transitory machine-readable medium for identifying usage information, in accordance with examples herein.

FIG. 10 is an example of a non-transitory machine-readable medium for identifying usage information with a remote device, in accordance with examples herein.

### DETAILED DESCRIPTION

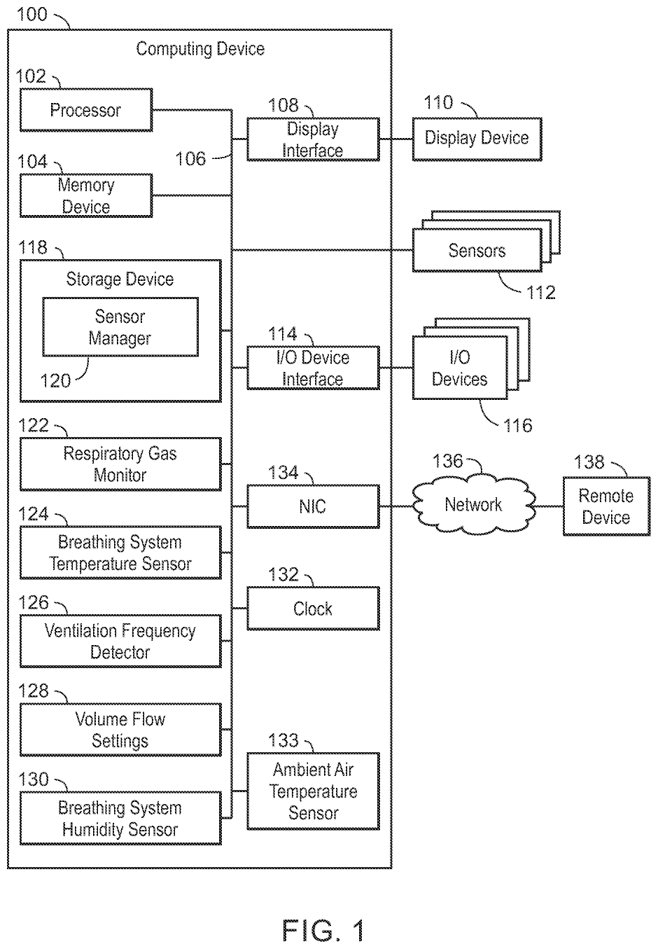

Embodiments of the present disclosure will now be described, by way of example, with reference to FIGS. 1-10, which relate to various embodiments of a system that facilitates identifying usage information for a flow sensor. For example, an anesthesia device can detect usage information from one or more flow sensors. The usage information, as referred to herein, includes data indicating a number of breathing cycles for a patient that have been monitored with a flow sensor. For example, the usage information can include a number of movements of a diaphragm of one or more flow sensors in a breathing circuit, a number of movements of any components of any other suitable sensors coupled to or incorporated within an anesthesia device, operating characteristics of an anesthesia device, or the like. The operating characteristics, as referred to herein, can include a flow rate of a flow sensor, a pressure differential of the flow sensor, a humidity of a breathing system, a temperature of the breathing system, a time stamp, a ventilation frequency, at least one flow setting, or the like.

The technical effect of identifying usage information for a sensor can include determining if a sensor, such as a flow sensor, among others, is to be repaired or replaced within a period of time. The present techniques have a technical advantage of enabling a device to obtain usage information associated with one or more sensors and determining whether any of the sensors have been used to monitor a number of breathing cycles that exceeds a predetermined limit. In some examples, the number of breathing cycles can be associated with multiple patients as the flow sensor can be coupled to multiple anesthesia devices over time. The present techniques can prevent a sensor from providing inaccurate information or malfunctioning by proactively installing a replacement sensor or repairing an existing sensor.

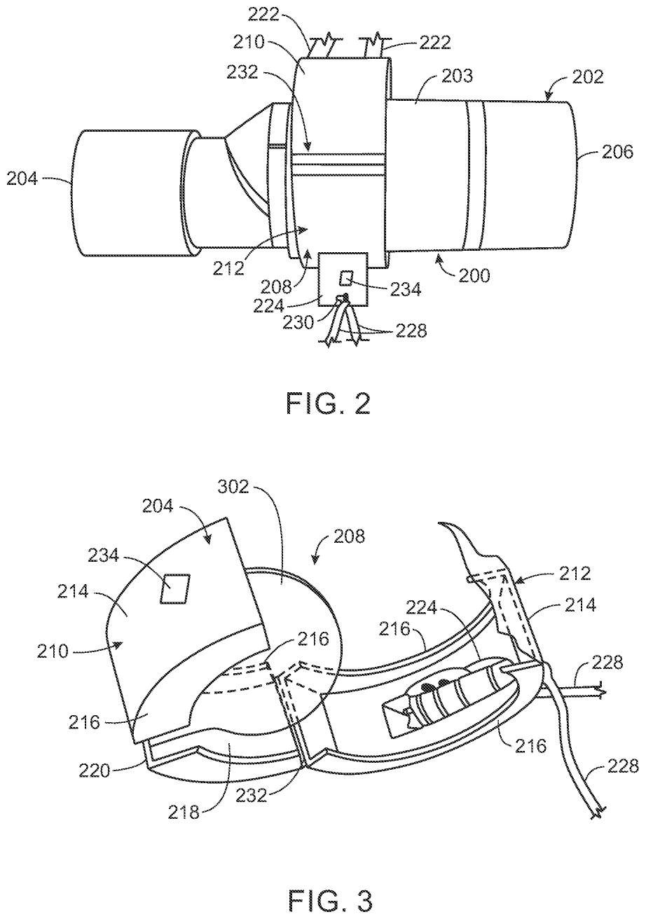

FIG. 1 is a block diagram of an example of a computing device that can identify usage information for a flow sensor. The computing device 100 may be, for example, a hospital monitor, an anesthesia device, an imaging device, such as an x-ray device or a magnetic resonance imaging device, a laptop computer, a desktop computer, a tablet computer, a mobile phone, or one or more servers providing a remote service, among others. The computing device 100 may include a processor 102 that is adapted to execute stored instructions, as well as a memory device 104 that stores instructions that are executable by the processor 102. The processor 102 can be a single core processor, a multi-core processor, a computing cluster, or any number of other configurations. The memory device 104 can include random access memory, read only memory, flash memory, or any other suitable memory systems. The instructions that are executed by the processor 102 may be used to implement a method that can identify usage information for a flow sensor, as described in greater detail below in relation to FIGS. 2-10.

The processor 102 may also be linked through the system interconnect 106 (e.g., PCI, PCI-Express, NuBus, etc.) to a display interface 108 adapted to connect the computing device 100 to a display device 110. The display device 110 may include a display screen that is a built-in component of the computing device 100. The display device 110 may also include a computer monitor, television, or projector, among others, that is externally connected to the computing device 100. The display device 110 can include light emitting diodes (LEDs), and micro-LEDs, Organic light emitting diode OLED displays, among others.

In some examples, one or more sensors 112 can be connected to the processor 102 using the system interconnect 106 or any other suitable interconnect and/or interface. The sensors 112 can include any number of flow sensors, such as variable orifice flow sensors, anesthesia related components, and the like. As described in greater detail below in relation to FIGS. 2 and 3, the sensors 112 can include storage devices (not depicted) that can store usage information, and predetermined limits for a maximum number of breathing cycles to be monitored with the sensors, among other data.

The processor 102 may be connected through a system interconnect 106 to an input/output (I/O) device interface 114 adapted to connect the computing device 100 to one or more I/O devices 116 The I/O devices 116 may include, for example, a keyboard and a pointing device, wherein the pointing device may include a touchpad or a touchscreen, among others. The I/O devices 116 may be built-in components of the computing device 100 or may be devices that are externally connected to the computing device 100.

In some embodiments, the processor 102 may also be linked through the system interconnect 106 to a storage device 118 that can include a hard drive, an optical drive, a USB flash drive, an array of drives, or any combinations thereof. In some embodiments, the storage device 118 can include any suitable applications. In some embodiments, the storage device 118 can include a sensor manager 120. In some embodiments, the sensor manager 120 can obtain usage information from a first flow sensor, such as sensor 112, coupled to the anesthesia device, such as computing device 100. The sensor manager 120 can also determine the usage information exceeds a predetermined limit, and generate an alert indicating the first flow sensor 112 is to be replaced.

In some examples, the sensor manager 120 can detect operating characteristics associated with gas flow for a patient from a respiratory gas monitor 122, a breathing system temperature sensor 124, a ventilation frequency detector 126, volume flow settings 128, a breathing system humidity sensor 130, and/or a clock 132. The sensor manager 132 can use the operating characteristics to determine a number of breathing cycles that a sensor 112 has monitored based on data from the respiratory gas monitor 122, the breathing system temperature sensor 124, the ventilation frequency detector 126, the volume flow settings 128, the breathing system humidity sensor 130, and/or the clock 132. In some examples, the clock 132 obtains or detects time stamps representing a time and/or date associated with data obtained from a sensor 112.

In some examples, the sensor manager 120 can also detect a first pressure differential from a first flow sensor and a second pressure differential from a second flow sensor. The sensor manager 120 can determine a difference between the first pressure differential and the second pressure differential and generate a condensation message indicating a presence of water condensation in the first flow sensor or the second flow sensor. For example, the sensor manager 120 can detect or obtain data from the breathing system temperature sensor 124, the breathing system humidity sensor 130, or a combination thereof, that indicates sensors 112 are operating in a high humidity environment. In response to detecting the difference between the pressure differentials from at least two sensors, the sensor manager 120 can determine that there is a presence of water in at least one of the sensors and that the condensation message is to be generated. In some examples, the sensor manager 120 can provide the condensation message to a user via the display device 110, haptic feedback, an audible alert, or the like.

In some examples, the processor 102 can also be linked through the system interconnect 106 to an ambient air temperature sensor 133. The ambient air temperature sensor 133 can sense the temperature proximate to the flow sensors, such as sensor 112, to detect a dew point value and condensation. In some examples, the ambient air temperature sensor 133 can enable determining if condensation has accumulated in a flow sensor based at least in part on the dew point, or any other value, obtained by the ambient air temperature sensor 133. For example, a high dew point can indicate that a malfunctioning flow sensor likely has condensation causing the detection of inaccurate sensor data.

In some examples, a network interface controller (also referred to herein as a NIC) 134 may be adapted to connect the computing device 100 through the system interconnect 106 to a network 136. The network 136 may be a cellular network, a radio network, a wide area network (WAN), a local area network (LAN), or the Internet, among others. The network 136 can enable data, such as alerts, messages, usage information, or predetermined limits for the sensors, among other data, to be transmitted from the computing device 100 to remote computing devices, remote display devices, and the like. For example, the network 136 may enable remote devices 138 to perform remote services and diagnostics related to the sensors 112.

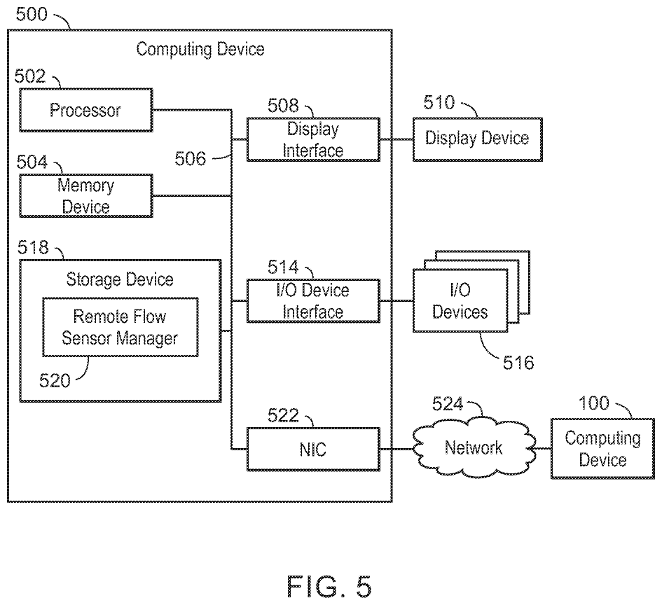

In some examples, a remote device 138 can receive data from the computing device 100. The remote device 138 can aggregate data from one or more computing devices and analyze usage information from multiple sensors 112, such as flow sensors. In some examples, the remote device 138 can obtain usage information from one or more flow sensors coupled to one or more anesthesia devices and determine a number of breathing cycles for each of the one or more flow sensors. The remote device 138 can also determine an accuracy limit value representing when the one or more flow sensors provide inaccurate flow values from an inhalation portion or an exhalation portion of a breathing circuit for a patient and transmit the accuracy limit to the one or more anesthesia devices, such as any number of computing devices 100. The remote device 138 is described in greater detail below in relation to FIG. 5.

It is to be understood that the block diagram of FIG. 1 is not intended to indicate that the computing device 100 is to include all of the components shown in FIG. 1. Rather, the computing device 100 can include fewer or additional components not illustrated in FIG. 1 (e.g., additional memory components, embedded controllers, additional modules, additional network interfaces, etc.). Furthermore, any of the functionalities of the sensor manager 120 may be partially, or entirely, implemented in hardware and/or in the processor 102. For example, the functionality may be implemented with an application specific integrated circuit, logic implemented in an embedded controller, or in logic implemented in the processor 102, among others. In some embodiments, the functionalities of the sensor manager 120 can be implemented with logic, wherein the logic, as referred to herein, can include any suitable hardware (e.g., a processor, among others), software (e.g., an application, among others), firmware, or any suitable combination of hardware, software, and firmware.

FIG. 2 illustrates an isometric view of a fluid flow sensor in accordance with some examples. Fluid flow sensor 200 is used to measure flow rates of fluids, such as moist gases, flowing through the fluid flow sensor 200, for example, by developing pressure differences within the fluid flow sensor 200 that are used to measure flow rates of the fluids. Fluid flow sensor 200 has a generally cylindrical configuration. However, fluid flow sensor 200 may be formed in a variety of shapes and sizes and still lie within the scope of this disclosure.

In one examples, the fluid flow sensor 200 can include a housing 202 that defines a fluid flow passage 203 with an inlet end 204 and an outlet end 206. When fluid flow sensor 200 is used for measuring gas flow rates in a breathing apparatus, the fluid flow sensor 200 can be inserted at one or more desired locations in a breathing circuit where the breathing gas is introduced into the housing 202 of the fluid flow sensor 200 through the inlet end 204 to pass through the fluid flow passage 203 and exit though the outlet end 206 to continue through the breathing circuit. The measurements made to determine the flow rate of the gas passing through the fluid flow sensor 200 are made as the gas passes through fluid flow passage 203 in the housing 202 from the inlet end 204 to the outlet end 206.

In some examples, a collar 208 is disposed around the housing 202 of the fluid flow sensor 200. Collar 208 can be configured to cover some or all of the exterior of the housing. In one examples, first portion 210 and second portion 212 of the collar 208 can each formed of a suitable material, such as a plastic material, and include an outer wall (214 of FIG. 3), a pair of side walls (216 of FIG. 3) extending along and outwardly from opposed sides of the outer wall (214 of FIG. 3) to define an interior (218 of FIG. 3) within the first portion 210 and second portion 212. The first portion 210 also defines a number of channels (220 of FIG. 3) that extend through the outer wall (214 of FIG. 3) and/or side walls (216 of FIG. 3) to enable tubes or hoses 222 or other items to be connected to the measurement ports (not depicted) on the housing 202 of the fluid flow sensor 200 through the first portion 210 to allow determination of the fluid flow rate of the gas passing through the sensor 200.

In some examples, the second portion 212 can be formed with a recess 224 extending outwardly from the outer wall (214 of FIG. 3) in a direction generally opposite the side walls (216 of FIG. 3). In one example, wires (228 of FIG. 3) can extend through apertures 230 in the recess 224 to a suitable power source and/or controller (not shown) for operation of electrical components.

In some examples, the first portion 210 and the second portion 212 are joined to one another at one end by a suitable connector (232 of FIG. 3). Connector 232 enables the first portion 210 and second portion 212 to be moved apart from one another to enable placement of the collar 208 around the housing 202 of the fluid flow sensor 200. The connector 232 can take any suitable shape or configuration, and can enable the first portion 210 and the second portion 212 to be completely separated from one another.

In some examples, the fluid flow sensor 200 can include a storage device 234 that can store usage information, predetermined limits, and any other suitable data associated with the fluid flow sensor. As discussed in greater detail below in relation to FIG. 3, the storage device 234 can store usage information indicating a number of times a diaphragm of the fluid flow sensor oscillates or otherwise moves during inhalation or exhalation by a patient. The storage device 234 can also store a predetermined limit representing a maximum number of breaths of a patient that can be monitored without the fluid flow sensor 200 losing accuracy or otherwise degrading or malfunctioning.

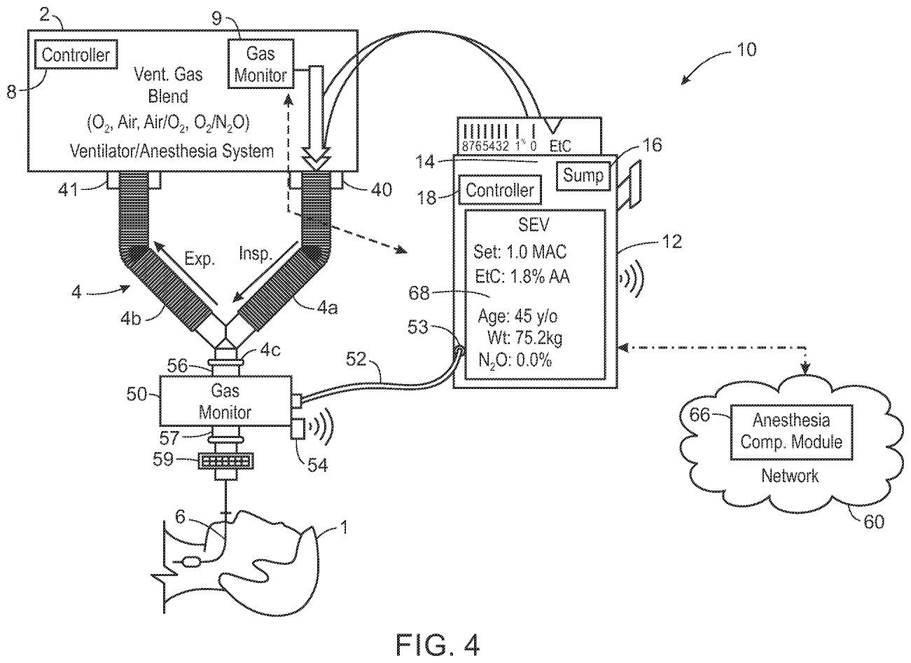

FIG. 3 depicts an example of an inside segment of a fluid flow sensor 200. In some examples, a diaphragm 302 can be located at any depth within the fluid flow sensor 200. The diaphragm 302 can oscillate or move as gases from a patient are inhaled through the fluid flow sensor 200 or exhaled through the fluid flow sensor 200. In some examples, a fluid flow sensor 200 can be placed within the inhalation portion of a breathing circuit for a patient and a separate fluid flow sensor 200 can be placed within the exhalation portion of the breathing circuit for the patient as described in greater detail below in relation to FIG. 4. The data from the fluid flow sensor 200 monitoring a patient can be detected, captured, or otherwise obtained by any number of wires, tubes, or hoses 212 that connect the flow sensors 200 to an anesthesia device, such as computing device 100 of FIG. 1. In some examples, the flow sensors 200 can include a wireless transmitter that can provide data, such as usage information, to an anesthesia device (not depicted) using any suitable wireless protocol such as Bluetooth®, Wi-Fi®, or the like.

FIG. 4 depicts an example of an electronic vaporizer system 10 operably connected to a ventilator system 2 and configured to deliver vaporized anesthetic agent to a patient breathing circuit 4. The electronic vaporizer system 10 includes an electronic vaporizer 12 and one or more sensors communicatively connected thereto, including a gas monitor 50 configured to measure end tidal concentration of the anesthetic agent in exhalation gasses from patient 1.

The electronic vaporizer 12 can include a sump 16, or reservoir, containing anesthetic agent to be delivered to the patient, such as Sevoflurane, Desflurane, Enflurane, etc. The sump 16 is configured to be refillable, such as from a refill bottle. Thus, the sump 16 has sufficient volume capacity such that it can receive at least the entire volume of a standard refill container. In one embodiment, the sump 16 can accommodate up to about 300 mL of liquid agent. The electronic vaporizer 12 includes a vaporizer unit 14 that vaporizes liquid anesthetic agent housed in a sump 16 and delivers the vaporized agent to the patient breathing circuit 4. For example, the breathing circuit 4 may include a patient breathing circuit 4, and the vaporizer unit 14 may be configured to deliver vaporized agent such that inhalation gasses comprises anesthetic agent are injected into the patient breathing circuit 4 and delivered to the patient 1 by the ventilator system 2.

The electronic vaporizer 12 further includes a controller 18 configured to control the vaporizer unit to deliver an amount of vaporized agent to maintain a desired end tidal concentration for the patient 1. The control system for the electronic vaporizer system includes the controller 18 for the vaporizer unit 14 and may also include other control devices communicatively connected to the controller 18. For example, the controller 18 may act in concert with an anesthesia computation module 66 on a network 60 communicatively connected to the electronic vaporizer 12 and/or a controller 8 for the ventilator system 2.

A gas sensor, which can include one or more flow sensors, is positioned to measure end tidal concentration of anesthetic agent and other gasses in exhalation gasses within the patient breathing circuit 4. The patient breathing circuit 4 includes an inspiratory flow sensor 40 and an inspiratory section 4a that carries inhalation gasses from the ventilator system to the patient interface 6. The expiratory section 4b is configured to carry exhalation gasses from the patient back to the ventilator 2 through the expiratory flow sensor 41. The patient interface is commonly, for example, an endotracheal tube. In other embodiments, the patient interface 6 may be a facial mask or some other device configured to create a sealed interface between the patient's airway and the breathing circuit 4. In the depicted example, the gas monitor 50 is positioned between the patient interface 6 and the inspiratory and expiratory arms of the patient breathing circuit 4. Humidity and moisture exchange filter 59 may be positioned between the patient interface 6 and the gas monitor 50 in order to remove moisture from the exhalation gasses prior to measurement.

The gas sensor is configured to measure concentration of the anesthetic agent in the exhalation gasses from the patient, and may also be configured to measure a concentration of nitrous oxide (N₂O) and carbon dioxide (CO₂) and oxygen (Od). Such concentration measurements are taken during the exhalation cycle where exhalation gasses exit the patient's lungs through the patient interface 6 through the filter 59 to the first connector end 57 of the unit containing the gas monitor 50 and out the second connector end 56, which is connected to the connector end 4c of the patient breathing circuit hose. The gas monitor 50 may further be configured to measure flowrate, including inspiratory flow rate and expiratory flow rate, as well as other gas concentration measurements, which may be inspiratory or expiratory measurements.

The concentration and other measurements from the gas monitor 50 are communicated to the electronic vaporizer 12, which may be by a physical data connection and/or by wireless means. In the example at FIG. 4, the gas monitor 50 is connected by cable 52 to the receiver port 53 on the electronic vaporizer 12. The gas monitor 50 also includes a wireless transmitter 54, which may be a wireless transceiver communication, which is configured to wirelessly broadcast the concentration and other measurements conducted by the gas monitor 50. Such wireless communications may be received by the network 60 such as the computer network system for the operating ward and/or by the hospital or healthcare facility network. In certain embodiments, the physical connection between the gas monitor 50 and the electronic vaporizer 12 may be eliminated and the electronic vaporizer 12 may be configured to receive wireless transmission of the measurements from the gas monitor 50.

An additional flow sensor, also referred to as gas monitor 9, may be configured to measures input gas from the ventilator to the patient's breathing circuit and configured to measure the ventilation gas blend provided by the ventilator 2. Such a gas monitor 9 may be positioned upstream of the delivery point from the vaporized agent and may be configured to measure flowrate and gas concentrations of the ventilator gas blend such as measurement of oxygen (O₂) and N₂O in the ventilator gas blend. This provides information regarding the input gasses and flow rates provided by the ventilator system. In certain embodiments, the gas monitor 9 may be incorporated in the ventilator system 2 and the gas measurements may be communicated by the ventilator system 2 to the electronic vaporizer 12. In other embodiments, the gas monitor 9 may be a stand-alone sensor connected at a point in the breathing circuit and configured to communicate directly with the electronic vaporizer 12, which may be by wired or wireless means as described above. The input gas concentration information may also be supplied by an electronic gas mixer built into the anesthesia machine, if so equipped. For example, the additional gas monitor 9 may be integrated into an electronic gas mixer that automatically blends and delivers mixed gas to the patient breathing circuit (N₂O/O₂, Air/O₂, O₂ or Air). In such an embodiment, the gas composition is obtained from the electronic gas mixer through wired or wireless communications as described herein.

In some examples, the electronic vaporizer 12 can include a user interface 68 that can provide any suitable data associated with a patient 1. For example, the user interface 68 can provide information about the patient 1, such as age and weight, as well as flow settings, and a temperature and humidity of a patient breathing circuit 4. The user interface 68 can also provide usage information for one or more flow sensors such as the gas sensors 9 and 50. As discussed above, any suitable usage information related to the gas sensors 9 and 50 can be provided by the user interface 68.

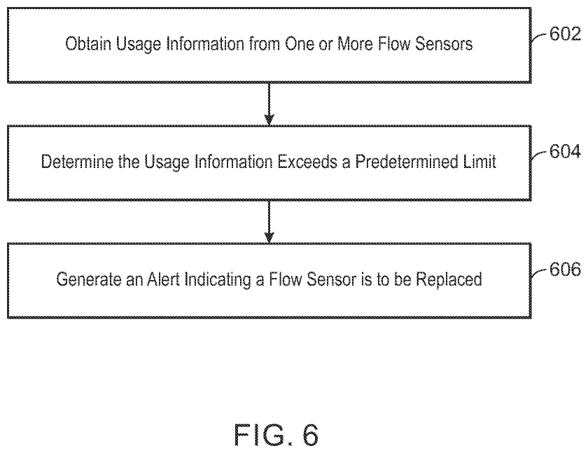

FIG. 5 depicts an example remote device electronically coupled to an anesthesia device. The remote device 500 may be, for example, one or more servers providing a remote service, a remote hospital monitor, an anesthesia device, an imaging device, such as an x-ray device or a magnetic resonance imaging device, a laptop computer, a desktop computer, a tablet computer, a mobile phone, or, among others. The remote device 500 may include a processor 502 that is adapted to execute stored instructions, as well as a memory device 504 that stores instructions that are executable by the processor 502. The processor 502 can be a single core processor, a multi-core processor, a computing cluster, or any number of other configurations. The memory device 504 can include random access memory, read only memory, flash memory, or any other suitable memory systems. The instructions that are executed by the processor 502 may be used to implement a method that can identify usage information for a flow sensor, as described in greater detail below in relation to FIGS. 6-10.

The processor 502 may also be linked through the system interconnect 506 (e.g., PCI, PCI-Express, NuBus, etc.) to a display interface 508 adapted to connect the remote device 500 to a display device 510. The display device 510 may include a display screen that is a built-in component of the remote device 500. The display device 510 may also include a computer monitor, television, or projector, among others, that is externally connected to the remote device 500. The display device 510 can include light emitting diodes (LEDs), and micro-LEDs, Organic light emitting diode OLED displays, among others.

The processor 502 may be connected through a system interconnect 506 to an input/output (I/O) device interface 514 adapted to connect the remote device 500 to one or more I/O devices 516 The I/O devices 516 may include, for example, a keyboard and a pointing device, wherein the pointing device may include a touchpad or a touchscreen, among others. The I/O devices 516 may be built-in components of the remote device 500 or may be devices that are externally connected to the remote device 500.

In some embodiments, the processor 502 may also be linked through the system interconnect 506 to a storage device 518 that can include a hard drive, an optical drive, a USB flash drive, an array of drives, or any combinations thereof. In some embodiments, the storage device 518 can include any suitable applications. In some embodiments, the storage device 518 can include a remote flow sensor manager 520. In some embodiments, the remote flow sensor manager 520 can receive data from the computing device 100. The remote flow sensor manager 520 can aggregate data from one or more computing devices 100 and analyze usage information from multiple sensors 112, such as flow sensors. In some examples, the remote flow sensor manager 520 can obtain usage information from one or more flow sensors coupled to one or more anesthesia devices and determine a number of breathing cycles for each of the one or more flow sensors, such as sensors 112 of FIG. 1. The remote flow sensor manager 520 can also determine an accuracy limit value representing when the one or more flow sensors, such as sensors 112 of FIG. 1, provide inaccurate flow values from an inhalation portion or an exhalation portion of a breathing circuit for a patient and transmit the accuracy limit to the one or more anesthesia devices, such as any number of computing devices 100.

In some examples, a network interface controller (also referred to herein as a NIC) 522 may be adapted to connect the computing device 100 of FIG. 1 through the system interconnect 506 to a network 524. The network 524 may be a cellular network, a radio network, a wide area network (WAN), a local area network (LAN), or the Internet, among others. The network 524 can enable data, such as alerts, among other data, to be transmitted from the remote device 500 to other computing devices, such as computing device 100 of FIG. 1, remote display devices, and the like.

It is to be understood that the block diagram of FIG. 5 is not intended to indicate that the remote device 500 is to include all of the components shown in FIG. 5. Rather, the remote device 500 can include fewer or additional components not illustrated in FIG. 5 (e.g., additional memory components, embedded controllers, additional modules, additional network interfaces, etc.). Furthermore, any of the functionalities of the remote flow sensor manager 520 may be partially, or entirely, implemented in hardware and/or in the processor 502. For example, the functionality may be implemented with an application specific integrated circuit, logic implemented in an embedded controller, or in logic implemented in the processor 502, among others. In some embodiments, the functionalities of the remote flow sensor manager 520 can be implemented with logic, wherein the logic, as referred to herein, can include any suitable hardware (e.g., a processor, among others), software (e.g., an application, among others), firmware, or any suitable combination of hardware, software, and firmware.

FIG. 6 illustrates a process flow diagram of an example method for identifying usage information of a flow sensor. In some examples, the method 600 can be implemented with any suitable computing system, such as the computing device 100 of FIG. 1, or the remote device 500 of FIG. 5, among others.

At block 602, the method 600 can include obtaining usage information from one or more flow sensors coupled to an anesthesia device. In some examples, the usage information can include a number of movements of a diaphragm of one or more flow sensors in a breathing circuit. The usage information can also include movements of any components of any other suitable sensors to or incorporated within an anesthesia device. In some examples, the usage information can also include operating characteristics of an anesthesia device. The operating characteristics can include a flow rate of a flow sensor, a pressure differential of the flow sensor, a humidity of a breathing system, a temperature of the breathing system, a time stamp, a ventilation frequency, at least one flow setting, or a combination thereof. In some examples, the usage information can also provide a differential pressure from one or more flow sensors. The differential pressure can be displayed via a display device, provided by an audio alert, or transmitted to a remote device.

At block 604, the method 600 can include determining the usage information exceeds a predetermined limit. For example, a flow sensor can include a storage device to store a predetermined limit as well as usage information such as a number of movements of a diaphragm of the flow sensor. The number of movements of the diaphragm, or any other component of a flow sensor, can indicate how many breathing cycles a flow sensor has measured or monitored, which can be compared to the predetermined limit. If the usage information exceeds the predetermined limit, the flow sensor has exceeded the expected number of measurements the flow sensor is designed to obtain or otherwise detect.

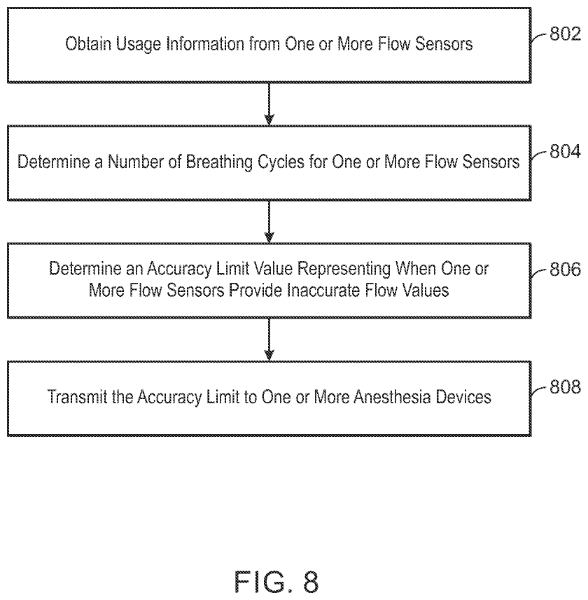

In some examples, the predetermined limit can be detected, received, or otherwise obtained from user input, from a remote device or application, or from any other suitable source. The predetermined limit can be a fixed value or a value that can be modified. For example, the predetermined limit can be set for a flow sensor and stored at the time of manufacturing the flow sensor or the predetermined limit can be modified as the maximum number of breathing cycles that a flow sensor can monitor is detected. In some examples, the maximum number of breathing cycles that a flow sensor can monitor can be adjusted as a set of flow sensors are in operation and are determined to accurately monitor a number of breathing cycles for a patient that exceeds an original predetermined limit. Techniques for adjusting or modifying the predetermined limit are described in greater detail below in relation to FIG. 8.

Still at block 604, in some examples, the method 600 can include obtaining the number of movements of a diaphragm from one or more flow sensors using any suitable computing device coupled to the flow sensors. For example, the flow sensors can periodically or continuously in real-time transmit data to a computing device indicating the number of movements of the diaphragms, or any other components, of the flow sensors.

At block 606, the method 600 can include generating an alert indicating a flow sensor is to be replaced. In some examples, an alert is not generated until the usage information exceeds the predetermined limit. If the usage information does exceed the predetermined limit, the alert can indicate a particular flow sensor in a breathing circuit is to be replaced. Otherwise, the method 600 can continue tracking the usage information. For example, two flow sensors may reside within a patient breathing circuit to monitor gases inhaled or exhaled by a patient. One of the flow sensors (a first flow sensor) may be new, while the other flow sensor (a second flow sensor) may have been coupled to the patient after monitoring patients with another anesthesia device. The alert can indicate that the second flow sensor has exceeded its predetermined limit based on usage information from the second flow sensor. In some examples, the anesthesia device can store usage information for each flow sensor along with unique identifiers, such as serial numbers or the like, for each flow sensor. The unique identifiers can enable the anesthesia device to identify the flow sensor to be replaced or repaired.

In some examples, the method 600 can also include providing the usage information via a display device. The usage information can be continuously displayed whether or not an alert has been generated. For example, the usage information can indicate a number of breathing cycles that a flow sensor has monitored in order to provide a user with data indicating approximately when a flow sensor will be replaced. In some examples, the method 600 can also include generating or calculating statistics indicating an estimated time that a flow sensor will exceed a predetermined limit based on operating characteristics for the flow sensor such as the ventilation rate, among others.

The process flow diagram of method 600 of FIG. 6 is not intended to indicate that all of the operations of blocks 602-606 of the method 600 are to be included in every example. Additionally, the process flow diagram of method 600 of FIG. 6 describes a possible order of executing operations. However, it is to be understood that the operations of the method 600 can be implemented in various orders or sequences. In addition, in some examples, the method 600 can also include fewer or additional operations.

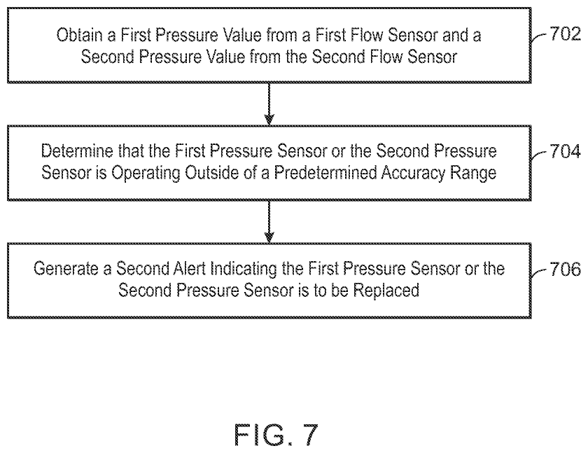

FIG. 7 is a process flow diagram of another example method for identifying usage information of a flow sensor. In some examples, the method 700 can be implemented with any suitable computing system, such as the computing device 100 of FIG. 1, or the remote device 500 of FIG. 5, among others. In some examples, the method 700 can be implemented with two or more flow sensors, such as the fluid flow sensor 200 of FIGS. 2 and 3.

At block 702, the method 700 can include obtaining a first pressure value from a first flow sensor and a second pressure value from the second flow sensor. In some examples, the method 700 can include obtaining the pressure values from any number of flow sensors coupled to an anesthesia device using any suitable wired or wireless protocol. For example, any number of flow sensors can be included within a breathing circuit for a patient to monitor inhaled gases and exhaled gases.

At block 704, the method 700 can include determining that the first pressure value and the second pressure value indicate that the first pressure sensor or the second pressure sensor is operating outside of a predetermined accuracy range. For example, the method 700 can include comparing the first pressure value and the second pressure value and determining that the difference between the pressure values is within a predetermined accuracy range. If the pressure values have a difference that is outside a predetermined accuracy range, the process flow continues at block 706. Otherwise, the method 700 can continue monitoring the pressure values obtained from the first flow sensor and the second flow sensor.

In some examples, the differential pressure across a diaphragm of a flow sensor is measured and used to correlate to a gas flow based on a predefined calibration curve of flow versus a differential pressure loaded into a flow sensor storage device, such as an EEPROM. The flow rate, such as mL/min, or any other measurement, integrated over time can be used to calculate the machine delivered inspiratory/expiratory volume. The flow sensor measured volumes can then be compared against the target delivery volume set by the user. As the flow sensor performance drifts due to diaphragm aging, condensation, gas composition changes, pressure sensor drift, or the like, the system calculated flow sensor delivery volume will, in some examples, correspondingly shift in magnitude. As the system tracks the deviation of the reported (as measured flow) against the target flow, the system can detect or determine that the measured accuracy is drifting under similar state conditions. For example, a system can detect, calculate, or otherwise determine how much the measured differential pressure has changed over time at the same user input targeted flow setting and use conditions based on a sensor local ambient temperature, inspiratory gas temperature, humidity, and pressure, among others.

At block 706, the method 700 can include generating a second alert indicating the first pressure sensor or the second pressure sensor is to be replaced. The second alert can include data indicating an identifier for the flow sensor to be replaced, the usage information for the flow sensor to be replaced, and the like. In some examples, the method 700 can include transmitting the second alert and the usage information for a first flow sensor and a second flow sensor to a remote device.

In some examples, the method 700 can also include detecting a first pressure differential from the first flow sensor and a second pressure differential from the second flow sensor and determining a difference between the first pressure differential and the second pressure differential. The method 700 can also include generating a condensation message indicating a presence of water condensation in the first flow sensor or the second flow sensor. For example, the method 700 can include obtaining the temperature and/or humidity of a breathing circuit for a patient and detecting that the pressure differential is likely causes to operation in a high humidity environment. In some examples, the method 700 can include providing the condensation message to a display device coupled to the anesthesia device, providing an audible alert representing the condensation message, or a combination thereof.

In some examples, during general anesthesia, two sources of heat and moisture in a circle breathing circuit can include the rebreathing of exhaled gas, and the water vapor and heat released from the CO2 absorbent in an exothermic reaction. The conservation of heat and moisture in the breathing circuit can depend on various factors including the Fresh Gas Flow (FGF), the breathing system configuration, and the operating room temperature. If the humidity and temperature of the breathing circuit gases are measured or known, and if the system also measures the ambient air temperature of the room, such as using the ambient air temperature sensor 133 of FIG. 1, the dew point can be calculated which can alert the system and user to potential condensation inside of a flow sensor. For example, if a workstation outlet inspiratory limb gas is 28 C (82.4 F) and 70% real humidity (typical values), the dewpoint would be at a room ambient temperature of 22.2 C (72 F). In some examples, operating rooms are maintained between 70 to 75 F (21 C to 24 C) with 50 to 60% relative humidity as a compromise between the requirements of the patients and those of the operators. Therefore, knowledge of the ambient temperature of the air immediately surrounding the flow sensors along with the inspiratory and expiratory limb percent real humidity can enable a system to predict condensation within a flow sensor. In some examples, a system can log the temperature and real humidity data and generate alerts in response to detecting an environment that produces condensation in the flow sensor.

In one example, if there is condensing moisture in a flow sensor, such as a variable orifice flow sensor, the presence of condensation decreases the response of the flow sensor by attenuating the deflection of the variable orifice membrane. This can require a high flow rate to attain the same differential pressure, which would be achieved without the condensate present. Likewise, if condensation accumulates in the pneumatic sensing tubes or causes occlusion of the very thin, laser cut aperture of the diaphragm of a flow sensor due to surface tension, then the differential pressure drop may spike on one flow sensor compared to anther flow sensor in the same breathing circuit. The difference in the differential pressure drop can trigger various flow sensor alarms such as “check flow”, “expiratory reverse flow”, inspiratory reverse flow”, and “Vte>Insp Vt”, among others.

The process flow diagram of method 700 of FIG. 7 is not intended to indicate that all of the operations of blocks 702-706 of the method 700 are to be included in every example. Additionally, the process flow diagram of method 700 of FIG. 7 describes a possible order of executing operations. However, it is to be understood that the operations of the method 700 can be implemented in various orders or sequences. In addition, in some examples, the method 700 can also include fewer or additional operations.

FIG. 8 is a process flow diagram of an example method for identifying accuracy information for a flow sensor. In some examples, the method 800 can be implemented with any suitable computing system, such as the computing device 100 of FIG. 1, or the remote device 500 of FIG. 5, among others.

At block 802, the method 800 can include obtaining usage information from one or more flow sensors coupled to one or more anesthesia devices. In some examples, the usage information can be obtained by an anesthesia device or computing device as discussed above in relation to block 602 of FIG. 6. The anesthesia device can transmit or forward the usage information to a remote device or the remote device can detect or otherwise obtain the usage information directly from the flow sensors.

At block 804, the method 800 can include determining a number of breathing cycles for one or more flow sensors. In some examples, the method 800 can include determining the number of breathing cycles for a patient by detecting a number of times a diaphragm or any other suitable portion of a flow sensor oscillates or otherwise moves in response to inhalation or exhalation by the patient. In some examples, the number of breathing cycles can be obtained from usage information from any number of flow sensors and the number of breathing cycles monitored by each flow sensor can be stored along with identifier information for the flow sensors and any suitable time stamp. The breathing cycles or any other suitable usage information for each flow sensor can be stored in a database or any other file format.

At block 806, the method 800 can include determining an accuracy limit value representing when one or more flow sensors provide inaccurate flow values from an inhalation portion or an exhalation portion of a breathing circuit for a patient. In some examples, the accuracy limit value can indicate when a flow sensor can no longer provide reliable data associated with the inhalation gases or exhalation gases of a patient's breathing circuit. For example, the accuracy limit value can be 1,000 breathing cycles, 10,000 breathing cycles, 100,000 breathing cycles, 1,000,000 breathing cycles, or any other suitable amount. A flow sensor may not be reliable when monitoring an inhalation or exhalation of a patient for a number of breathing cycles that exceeds the accuracy limit value.

In some examples, the accuracy limit value can be determined by analyzing data from a set of flow sensors. For example, the method 800 can include detecting or otherwise obtaining data from a set of flow sensors connected to any number of anesthesia devices. The data from the flow sensors can indicate the detected data from the flow sensors is outside of a predetermined range. For example, a flow in liters per minute detected by a first flow sensor may not match the flow in liters per minute detected by a second flow sensor. The mismatch in flow data detected by two flow sensors in a breathing circuit connected to a patient can indicate that at least one of the two flow sensors is incorrectly measuring or monitoring the inhalation gases provided to a patient or the exhalation gases received from the patient. The usage information indicating a number of breathing cycles that have been monitored by the flow sensors, one or more time stamps associated with the breathing cycles, and the mismatch of flow sensor data can be transmitted to an anesthesia device and/or a remote computing device for analysis using method 800. The accuracy limit value can be calculated by aggregating the usage information, flow sensor data, and time stamps from multiple flow sensors and determining a minimum number of breathing cycles that can be monitored before a flow sensor has an increased likelihood for providing inaccurate flow sensor data or measurements.

At block 808, the method 800 can include transmitting the accuracy limit to one or more anesthesia devices. In some examples, the accuracy limit can replace a predetermined limit stored in the anesthesia devices or the flow sensors. For example, a flow sensor in a breathing circuit can include a storage device to store a predetermined limit representing a number of breathing cycles that the flow sensor can monitor before being replaced. In some examples, the accuracy limit value can replace the predetermined limit if the method 800 determines that a flow sensor can monitor fewer or additional breathing cycles of a patient without degradation of the accuracy of the flow sensor.

The process flow diagram of method 800 of FIG. 8 is not intended to indicate that all of the operations of blocks 802-808 of the method 800 are to be included in every example. Additionally, the process flow diagram of method 800 of FIG. 8 describes a possible order of executing operations. However, it is to be understood that the operations of the method 800 can be implemented in various orders or sequences. In addition, in some examples, the method 800 can also include fewer or additional operations.

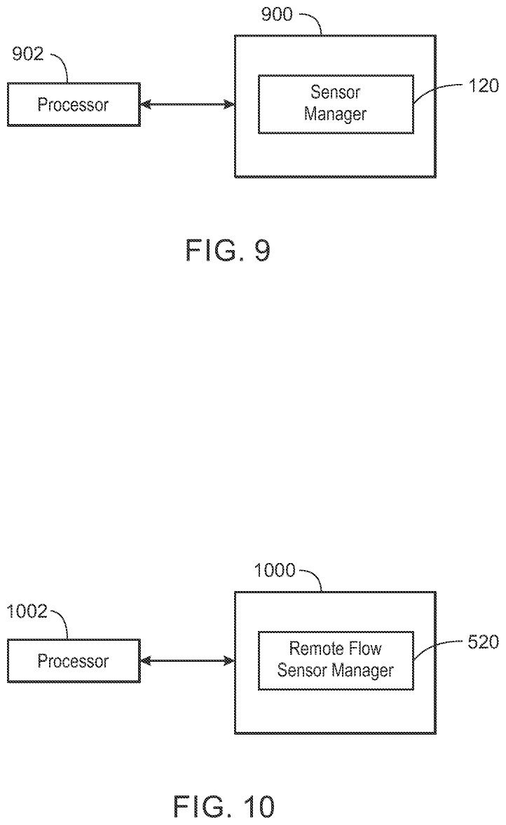

FIG. 9 is an example of a non-transitory machine-readable medium for identifying usage information, in accordance with examples herein. The non-transitory, machine-readable medium 900 can cause a processor 902 to implement the functionalities of methods 600, 700, and 800. For example, a processor of a computing device (such as processor 102 of FIG. 1 or processor 502 of FIG. 5), can access the non-transitory, machine-readable media 900.

In some examples, the non-transitory, machine-readable medium 900 can include instructions to execute a sensor manager 120. For example, the non-transitory, machine-readable medium 900 can include instructions for the sensor manager 120 that cause the processor 902 to obtain usage information from a first flow sensor coupled to an anesthesia device, determine the usage information exceeds a predetermined limit, and generate an alert indicating the first flow sensor is to be replaced.

In some examples, the sensor manager 120 can also cause the processor 902 to detect a first pressure differential from a first flow sensor and a second pressure differential from a second flow sensor. The sensor manager 120 can also determine a difference between the first pressure differential and the second pressure differential and generate a condensation message indicating a presence of water condensation in the first flow sensor or the second flow sensor. In some examples, the non-transitory, machine-readable medium 900 can include instructions to implement any combination of the techniques of the method 600, 700, and 800 described above.

FIG. 10 is an example of a non-transitory machine-readable medium for identifying usage information with a remote device, in accordance with examples herein. The non-transitory, machine-readable medium 1000 can cause a processor 1002 to implement the functionalities of methods 600, 700, and 800. For example, a processor of a computing device (such as processor 102 of FIG. 1 or processor 502 of FIG. 5), can access the non-transitory, machine-readable media 1000.

In some examples, the non-transitory, machine-readable medium 1000 can include instructions to execute a remote flow sensor manager 520. For example, the non-transitory, machine-readable medium 1000 can include instructions for the remote flow sensor manager 520 that cause the processor 1002 to aggregate data from one or more computing devices and analyze usage information from multiple sensors, such as flow sensors. In some examples, the remote flow sensor manager 520 can obtain usage information from one or more flow sensors coupled to one or more anesthesia devices and determine a number of breathing cycles for each of the one or more flow sensors. The remote flow sensor manager 520 can also determine an accuracy limit value representing when the one or more flow sensors provide inaccurate flow values from an inhalation portion or an exhalation portion of a breathing circuit for a patient and transmit the accuracy limit to the one or more anesthesia devices.

In some examples, the non-transitory, machine-readable medium 1000 can include instructions to implement any combination of the techniques of the method 600, 700, and 800 described above.

### Examples

In some examples, an anesthesia device can include a processor that can obtain usage information from a first flow sensor coupled to the anesthesia device and determine the usage information exceeds a predetermined limit. The processor can also generate an alert indicating the first flow sensor is to be replaced. The usage information can include a number of movements of a diaphragm of the first flow sensor.

Alternatively, or in addition, the usage information can further include operating characteristics of the anesthesia device, wherein the operating characteristics comprise a flow rate of the first flow sensor, a pressure differential of the first flow sensor, a humidity of a breathing system that includes the first flow sensor, a temperature of the breathing system, a time stamp, a ventilation frequency, at least one flow setting, or a combination thereof. Alternatively, or in addition, the first flow sensor comprises a storage device to store a number of movements of a diaphragm of the first flow sensor.

Alternatively, or in addition, the processor can provide the alert to a display device coupled to the anesthesia device or to a remote device. Alternatively, or in addition, the anesthesia device can include a second flow sensor, wherein the processor can obtain a first pressure value from the first flow sensor and a second pressure value from the second flow sensor, determine that the first pressure value and the second pressure value indicate that the first pressure sensor or the second pressure sensor is operating outside of a predetermined accuracy range, and generate a second alert indicating the first pressure sensor or the second pressure sensor is to be replaced.

Alternatively, or in addition, the processor can transmit the usage information for the first flow sensor and a second flow sensor to a remote device. Alternatively, or in addition, the processor can detect a first pressure differential from the first flow sensor and a second pressure differential from the second flow sensor, determine a difference between the first pressure differential and the second pressure differential is due to condensation, and generate a condensation message indicating a presence of water condensation in the first flow sensor or the second flow sensor.

Alternatively, or in addition, the processor can provide the condensation message to a display device coupled to the anesthesia device, provide an audible alert representing the condensation message, or a combination thereof. Alternatively, or in addition, the processor can provide a differential pressure from the first flow sensor via a display device.

Alternatively, or in addition, the processor can provide the usage information via a display device, wherein the usage information comprises a number of movements of a diaphragm of the first flow sensor.

In one aspect, a device can include a processor to obtain usage information from one or more flow sensors coupled to one or more anesthesia devices, determine a number of breathing cycles monitored by each of the one or more flow sensors, determine an accuracy limit value representing when the one or more flow sensors provide inaccurate flow values from an inhalation portion or an exhalation portion of a breathing circuit for a patient, and transmit the accuracy limit to the one or more anesthesia devices.

Alternatively, or in addition, the anesthesia device can provide a message indicating at least one of the flow sensors coupled to the anesthesia device is to be replaced due to the at least one of the flow sensors exceeding the accuracy limit value. Alternatively, or in addition, the processor can track the usage information from the one or more flow sensors using a unique identifier associated with each of the one or more flow sensors. Alternatively, or in addition, the processor can track the usage information from the one or more flow sensors as the one or more flow sensors are coupled to a first anesthesia device and as the one or more flow sensors are coupled to a second anesthesia device.

In one aspect, a method for detecting usage information from sensors can include obtaining usage information from a first flow sensor coupled to the anesthesia device, wherein the usage information comprises a number of movements of a diaphragm of the first flow sensor, determining the usage information exceeds a predetermined limit, and generating an alert indicating the first flow sensor is to be replaced.

Alternatively, or in addition, the usage information can include operating characteristics of the anesthesia device, wherein the operating characteristics comprise a flow rate of the first flow sensor, a pressure differential of the first flow sensor, a humidity of a breathing system that includes the first flow sensor, a temperature of the breathing system, a time stamp, a ventilation frequency, at least one flow setting, or a combination thereof.

Alternatively, or in addition, the first flow sensor can include a storage device to store the number of movements of a diaphragm of the first flow sensor. Alternatively, or in addition, the method can include obtaining a first pressure value from the first flow sensor and a second pressure value from a second flow sensor, determining that the first pressure value and the second pressure value indicate that the first pressure sensor or the second pressure sensor is operating outside of a predetermined accuracy range, and generating a second alert indicating the first pressure sensor or the second pressure sensor is to be replaced.

Alternatively, or in addition, the method can include detecting a first pressure differential from the first flow sensor and a second pressure differential from the second flow sensor, determining a difference between the first pressure differential and the second pressure differential is due to condensation, and generating a condensation message indicating a presence of water condensation in the first flow sensor or the second flow sensor.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

What is claimed is:

1. An anesthesia device comprising:
a processor to:
obtain usage information from a first flow sensor coupled to the anesthesia device;
determine the usage information exceeds a predetermined limit; and
generate an alert indicating the first flow sensor is to be replaced.

2. The anesthesia device of claim 1, wherein the usage information comprises a number of movements of a diaphragm of the first flow sensor.

3. The anesthesia device of claim 2, wherein the usage information further comprises operating characteristics of the anesthesia device, wherein the operating characteristics comprise a flow rate of the first flow sensor, a pressure differential of the first flow sensor, a humidity of a breathing system that includes the first flow sensor, a temperature of the breathing system, a time stamp, a ventilation frequency, at least one flow setting, or a combination thereof.

4. The anesthesia device of claim 1, when the first flow sensor comprises a storage device to store a number of movements of a diaphragm of the first flow sensor.

5. The anesthesia device of claim 1, wherein the processor is to provide the alert to a display device coupled to the anesthesia device or to a remote device.

6. The anesthesia device of claim 1, further comprising a second flow sensor, wherein the processor is to:
obtain a first pressure value from the first flow sensor and a second pressure value from the second flow sensor;
determine that the first pressure value and the second pressure value indicate that the first pressure sensor or the second pressure sensor is operating outside of a predetermined accuracy range; and
generate a second alert indicating the first pressure sensor or the second pressure sensor is to be replaced.

7. The anesthesia device of claim 1, wherein the processor is to transmit the usage information for the first flow sensor and a second flow sensor to a remote device.

8. The anesthesia device of claim 1, wherein the processor is to:
detect a first pressure differential from the first flow sensor and a second pressure differential from the second flow sensor;
determine a difference between the first pressure differential and the second pressure differential is due to condensation; and
generate a condensation message indicating a presence of water condensation in the first flow sensor or the second flow sensor.

9. The anesthesia device of claim 8, wherein the processor is to provide the condensation message to a display device coupled to the anesthesia device, provide an audible alert representing the condensation message, or a combination thereof.

10. The anesthesia device of claim 1, wherein the processor is to provide a differential pressure from the first flow sensor via a display device.

11. The anesthesia device of claim 1, wherein the processor is to provide the usage information via a display device, wherein the usage information comprises a number of movements of a diaphragm of the first flow sensor.

12. A device comprising a processor to:
obtain usage information from one or more flow sensors coupled to one or more anesthesia devices;
determine a number of breathing cycles monitored by each of the one or more flow sensors;
determine an accuracy limit value representing when the one or more flow sensors provide inaccurate flow values from an inhalation portion or an exhalation portion of a breathing circuit for a patient; and
transmit the accuracy limit to the one or more anesthesia devices.

13. The device of claim 12, wherein the anesthesia device is to provide a message indicating at least one of the flow sensors coupled to the anesthesia device is to be replaced due to the at least one of the flow sensors exceeding the accuracy limit value.

14. The device of claim 12, wherein the processor is to track the usage information from the one or more flow sensors using a unique identifier associated with each of the one or more flow sensors.

15. The device of claim 14, wherein the processor is to track the usage information from the one or more flow sensors as the one or more flow sensors are coupled to a first anesthesia device and as the one or more flow sensors are coupled to a second anesthesia device.

16. A method for detecting usage information of a flow sensor comprising:
obtaining the usage information from a first flow sensor coupled to the anesthesia device, wherein the usage information comprises a number of movements of a diaphragm of the first flow sensor;
determining the usage information exceeds a predetermined limit; and
generating an alert indicating the first flow sensor is to be replaced.

17. The method of claim 16, wherein the usage information further comprises operating characteristics of the anesthesia device, wherein the operating characteristics comprise a flow rate of the first flow sensor, a pressure differential of the first flow sensor, a humidity of a breathing system that includes the first flow sensor, a temperature of the breathing system, a time stamp, a ventilation frequency, at least one flow setting, or a combination thereof.

18. The method of claim 16, when the first flow sensor comprises a storage device to store the number of movements of a diaphragm of the first flow sensor.

19. The method of claim 16, further comprising:
obtaining a first pressure value from the first flow sensor and a second pressure value from a second flow sensor;
determining that the first pressure value and the second pressure value indicate that the first pressure sensor or the second pressure sensor is operating outside of a predetermined accuracy range; and
generating a second alert indicating the first pressure sensor or the second pressure sensor is to be replaced.

20. The method of claim 16, further comprising:
detecting a first pressure differential from the first flow sensor and a second pressure differential from the second flow sensor;
determining a difference between the first pressure differential and the second pressure differential is due to condensation; and
generating a condensation message indicating a presence of water condensation in the first flow sensor or the second flow sensor.
